(19) United States
(12) Patent Application Publication
(10) Pub. No.: US 2009/0326219 A1
(43) Pub. Date: Dec. 31, 2009

(54) PROCESS FOR MANUFACTURING EXTREMELY PURE BENZAZEPINE DERIVATIVES

(75) Inventors: Klaus GERDES, Dusseldorf (DE); József GUNGL, Sopron (HU); Beate KÄLZ, Steinbrunn (AT); Jan ROTHENBURGER, Oslip (AT); Stefan WELZIG, Wien (AT)

Correspondence Address: YOUNG & THOMPSON, 209 Madison Street, Suite 500, Alexandria, VA, 22314, US

(73) Assignee: SANOCHEMIA PHARMAZEUTIKA AG, Wien (AT)

(21) Appl. No.: 12/492,686

(22) Filed: Jun. 26, 2009

(30) Foreign Application Priority Data
Jun. 26, 2008 (AT) A 1021/2008

(51) Int. Cl. C07D 491/06

(52) U.S. Cl. 540/581

(57) ABSTRACT

A process for the production of extremely pure galanthamine or extremely pure galanthamine derivatives, a start is made from racemic bromine narwedine, which is debrominated under palladium catalysis. In this case, the working-up of the reaction mixture, which is carried out in the presence of oxygen or peroxides so that the palladium catalyst in an insoluble form is converted into an easily separable form, is essential. The further reaction is carried out by reduction of enantiomer-pure narwedine to form enantiomer-pure galanthamine, whereby it is then alkylated or dealkylated so that a corresponding substitution on the ring-nitrogen atom is achieved. By further purification, such as recrystallization, residual portions of palladium of below 5 ppm are achieved, so that direct use as a pharmaceutical raw material is made possible.

The invention relates to a process for the production of extremely pure 4a,5,9,10,11,12-hexahydro-6H-benzofuro[3a,3,2-ef][2]benzazepine derivatives of general formula I

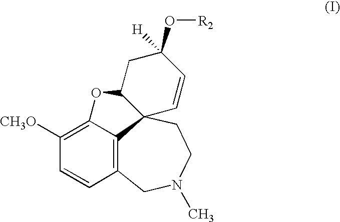

of formula IA

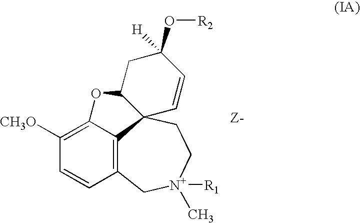

and of formula II

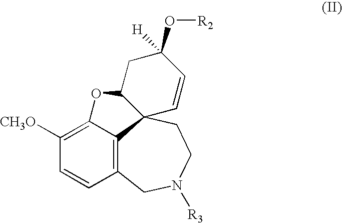

in which R1 is selected from the group that consists of hydrogen, hydroxy, alkoxy, low alkyl(C2-C10), which optionally is substituted by at least one halogen, low alkenyl(C2-C10), aryl, aralkyl, aryloxyalkyl; R2 is selected from the group that consists of low alkyl (C2-C10), which optionally is substituted by at least one halogen, low alkenyl(C2-C10), low alkinyl(C2-C10), aryl, aralkyl, aryloxyalkyl, alkylcarbonyl, arylcarbonyl, aralkylcarbonyl, alkyloxycarbonyl, aryloxycarbonyl, aralkyloxycarbonyl, alkylthionyl, arylthionyl, aralkylthionyl, alkyloxythionyl, aryloxythionyl, aralkyloxythionyl, alkylsulfonyl, aralkylsulfonyl, arylsulfonyl, carboxamide, thiocarboxamide; R3 is selected from the group that consists of hydrogen, hydroxy, alkoxy, low alkyl(C2-C10), which optionally is substituted by at least one halogen, low alkenyl(C2-C10), low alkinyl (C2-C10), aryl, aralkyl, aryloxyalkyl, formyl, alkylcarbonyl, arylcarbonyl, aralkylcarbonyl, alkyloxycarbonyl, aryloxycarbonyl, aralkyloxycarbonyl, alkylsulfonyl, aralkylsulfonyl, arylsulfonyl, and whereby Z is an anion of a pharmaceutically acceptable organic acid or an inorganic anion.

Galanthamine is an alkaloid with high pharmacological activity that primarily occurs in plants of the Amaryllidaceae type. In particular, its action as a more selective acetylcholinesterase inhibitor and the associated application in the treatment of neurodegenerative diseases, such as Alzheimer's disease, are to be emphasized. The amounts isolated from the naturally occurring Caucasian snowdrops Galanthus Woronoyi are not sufficient, however, to meet the needs of a pharmaceutical raw material. Since the end of the 1960s, therefore, galanthamine syntheses have been known that occasionally show, however, long and uneconomical reaction routes with poor total yields.

According to WO-A-97/110777, a more economical route for the galanthamine synthesis is to be provided by specific selection of bromine narwedine as a starting product if it is debrominated as bromine narwedine with palladium(II) acetate with the addition of triphenylphosphine. The racemic narwedine that is obtained contains about 700-800 ppm of palladium, however, which cannot be separated even after repeated treatment with activated carbon. Also, in additional reaction steps, such as the reduction of racemic narwedine, which is described according to WO-A-96/12692 of the applicant, palladium is further detected in the reaction end product despite repeated working-up. Galanthamine or galanthamine derivatives, which have palladium in a magnitude of 700-800 ppm, are not suitable, however, for the production of pharmaceutical agents, such as means for the treatment of Alzheimer's disease, since in the organism, produced by the palladium radicals, undesirable side effects can occur. Consequently, boundary values at <5 ppm are standardized for the oral administration of pharmaceutical agents, see “Note for Guidance on Specification Limits for Residues of Metal Catalysts” CPMP/SWP/QWP/4446/00.

The object of the invention is therefore to indicate a process of the above-mentioned type with which the above-mentioned, standardized boundary values for galanthamine derivatives of Formula I can be maintained.

According to the invention, a process for the production of the above-mentioned compounds with general Formula (I) is proposed, whereby racemic bromine narwedine (III) is debrominated with palladium(II) acetate and triphenylphosphine in a reaction step 1; the reaction mixture that contains racemic narwedine (IV) under oxygen contact or addition of peroxides is worked up in a reaction step 2 and converted into enantiomer-pure narwedine (V); and whereby enantiomer-pure galanthamine of general formula (VI) is obtained in a reaction step 3 by reduction; and compounds of general formula (I) are obtained in a reaction step 4 by O-alkylation or compounds of general formula (IA) are obtained in a reaction step 4′ by O-alkylation as well as subsequent salt formation, or compounds of general formula (II) are obtained in a reaction step 4″ by O-alkylation, N-demethylation and N-alkylation.

As an alternative, according to the invention, a process for the production of the above-mentioned compounds with general formula (I) or (II) is proposed, whereby in a reaction step 1, racemic bromine narwedine (III) is debrominated with palladium (II) acetate and triphenylphosphine; in a reaction step 2, the reaction mixture, containing racemic narwedine (IV), is worked up with use of peroxides and is converted into enantiomer-pure narwedine (V); and whereby in a reaction step 3, enantiomer-pure galanthamine of formula (VI) is obtained by reduction; and in a reaction step 4, compounds of general formula (I) are obtained by O-alkylation or in a reaction step 4′, compounds of general formula (IA) are obtained by O-alkylation as well as subsequent salt formation, or in a reaction step 4″, compounds of general formula (II) are obtained by O-alkylation, N-demethylation and N-alkylation.

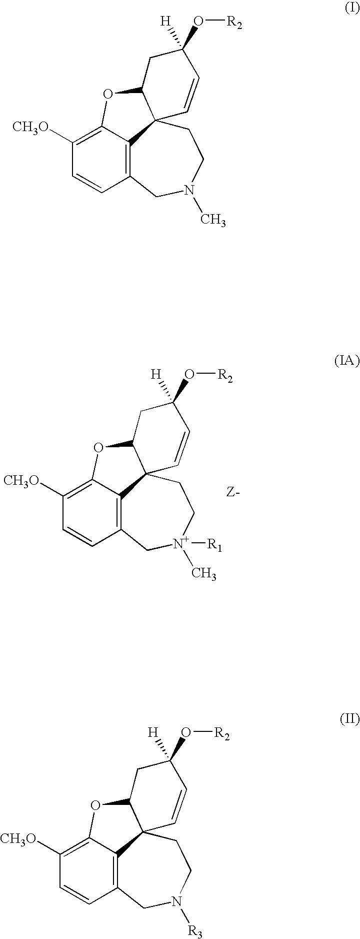

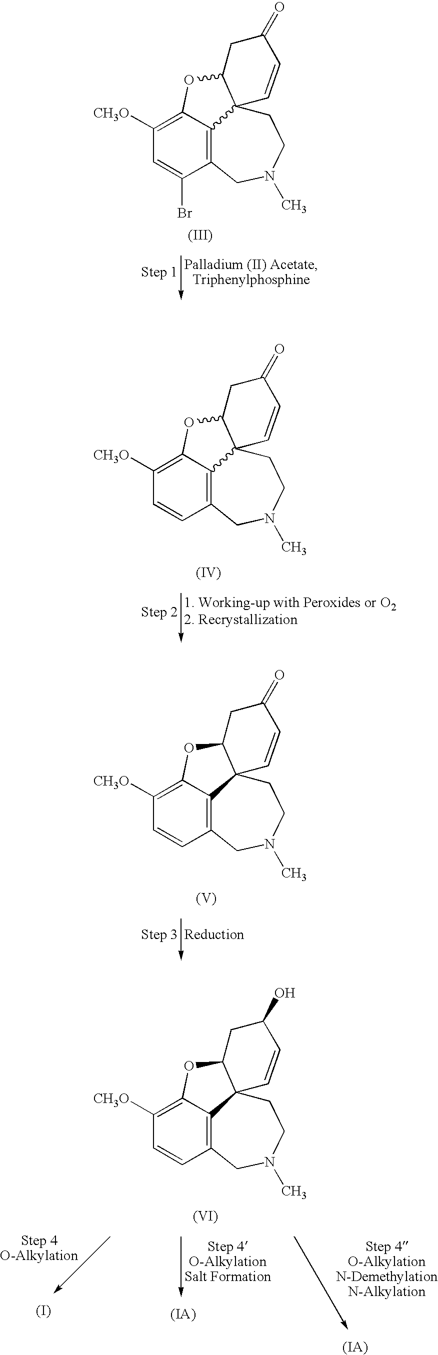

Advantageous configurations of the process according to the invention are subjects of the subclaims.

The invention is explained in more detail below based on embodiments for implementing the invention, whereby reference is made to the process steps according to the reaction diagram.

Step 1: Racemic bromine narwedine of general formula (III) is taken up in DMF and mixed with NaCO2H, PPH3, palladium (II) acetate as well as sodium hydroxide. This reaction mixture is heated to 94° C. and kept for six hours at this temperature, whereby the course of the reaction is tracked by means of chromatography. Then, the reaction mixture is worked up, whereby DMF is distilled off, and the racemic narwedine (IV) is precipitated by adding water and separated.
Step 2.1: The racemic narwedine (IV) that is obtained is taken up in a mixture of ethanol/triethylamine and mixed with activated carbon and a filter adjuvant. The mixture is refluxed for one to four hours while being stirred intensively, whereby an air-nitrogen mixture is blown through the reactor with, for example, 5% by volume of oxygen. It was found, surprisingly enough, that by the treatment with activated carbon, on the one hand, and the oxygen contact, on the other hand, the reduction of the palladium portions of significantly more than 95% in comparison to known, detectable palladium portions could be achieved. This is to be explained in more detail based on the following table:

| | 1^{st} Feedstock | 2^{nd} Feedstock | 3^{rd} Feedstock |
|---|---|---|---|
| | Pd (ppm) | Pd (ppm) | Pd (ppm) |
| Racemic Narwedine | 813 | 748 | 753 |
| (−)-Narwedine | 24 | 26 | 14 |

From this list in tabular form, it can be seen that palladium radicals of 748 to 813 ppm can be detected in the racemic narwedine mixture. Reaction end products with these proportions of palladium are unsuitable for a further use for the production of a pharmaceutical agent. By the working-up of the reaction mixture with activated carbon according to the invention with simultaneous oxygen contact, the palladium catalyst is converted into an insoluble, oxidized form, so that a separation in a ppm range of less than 26, preferably less than 24, especially preferably less than 14, is possible.

In an alternative process variant, the racemic narwedine (IV) that is obtained is also taken up in a mixture of 30 ethanol/triethylamine and mixed with activated carbon and a filter adjuvant; however, this mixture is then slowly mixed with 0.1-1% by weight of hydrogen peroxide while being stirred intensively and refluxed for one to four hours. Surprisingly enough, it was also found in this process variant that by the treatment with activated carbon, on the one hand, and the use of hydrogen peroxide, on the other hand, the palladium portion could be significantly reduced after filtration in comparison to known, detectable palladium portions. The measured values can be seen in the following table:

| | 1^{st} Feedstock | 2^{nd} Feedstock | 3^{rd} Feedstock |
|---|---|---|---|
| | Pd (ppm) | Pd (ppm) | Pd (ppm) |
| Racemic Narwedine | 800 | 810 | 763 |
| (−)-Narwedine | 22 | 24 | 16 |
| (H2O2-Treated) | | | |

In another process variant, the mixture that consists of racemic narwedine (IV), ethanol, triethylamine, activated carbon and filter adjuvant is mixed with 0.1-1% by weight of metachloroperbenzoic acid while being stirred intensively and refluxed for one to four hours. Also, in this process variant, it was found, surprisingly enough, that by the treatment with activated carbon, on the one hand, and the use of metachloroperbenzoic acid, on the other hand, the palladium portion could be significantly reduced after filtration in comparison to known, detectable palladium portions. The determined values are cited in the table below:

| | 1^{st} Feedstock | 2^{nd} Feedstock | 3^{rd} Feedstock |
|---|---|---|---|
| | Pd (ppm) | Pd (ppm) | Pd (ppm) |
| Racemic Narwedine | 778 | 805 | 767 |
| (−)-Narwedine | 20 | 23 | 18 |
| (MCPBA-Treated) | | | |

Step 2.2: The reaction mixture that is obtained according to Step 2.1 is cooled and inoculated with (−)narwedine crystals, so that enantiomer-pure (−)narwedine with general formula (V) is obtained.
Step 3: The enantiomer-pure (−)narwedine with general formula (V) that is obtained after recrystallization is, as described in WO-A-96/12692, mixed with a one-molar 5 solution of L-selectride in THF, allowed to stir for one hour, mixed with ethanol, and concentrated by evaporation. By the enantiomer-selective reduction, enantiomer-pure galanthamine of general formula (VI) is obtained. By recrystallization that is repeated one or more times, residual portions of palladium of less than 5 ppm are achieved. Therefore, by being worked up with oxygen or peroxide according to synthesis step 2.1, the palladium catalyst is converted into an insoluble oxidized form that can be easily separated by recrystallization during the course of the purification.
Step 4: The compound of general formula (VI) can be subjected to an O-alkylation in order to insert the radicals R2 into the oxygen atom.

### EXAMPLE

### SPH-1313

10 g of galanthamine is dissolved in 100 ml of pyridine, and acetyl chloride is slowly added at 25° C. It is stirred for 5 hours at room temperature and for 5 hours at 50° C. Then, the pyridine is spun off, and the residue is taken up in water and shaken out with ethyl acetate. The organic phase is spun in, and the crude product is recrystallized from ethanol. Yield 43.6%.

The measured palladium content was <5 ppm.

Step 4′: Step 4′ is carried out analogously to Step 4 with the difference that another reaction with an acid, such as, for example, hydrobromide, is carried out to form pharmaceutically acceptable salts with counter-anions Z—such as, for example, a bromide.
Step 4″: The compound of formula (VI) can be subjected to an N-demethylation with subsequent N- and O-derivatization.

### EXAMPLE SPH-1297

1.0 ml of vinyl chloroformate and 1.2 g of 1,8-bis(dimethylamino)naphthalene are added under protective atmosphere to a solution of 1 g of galanthamine in 50 ml of dichloromethane. The reaction mixture is stirred for 18 hours at 65° C., the solvent is distilled off, and the crude product is recrystallized from ethanol.

Yield 82.0%

The measured palladium content was <5 ppm.

The compounds with the general formula (I), (IA) or (II) can, if necessary, be further purified by recrystallization, so that a residual portion of less than 5 ppm is achieved. The above-mentioned embodiments were implemented such that R2 shows a substituent pattern, in which R2 represents carbonyl, carbonyloxy group and carboxamide. This exemplary selection, however, cannot be considered as a limitation of the scope of protection. The pharmacological action of the compounds according to general formulas (I), (IA) and (II) can be substantiated based on the measured IC50 values, since the latter represent any concentrations in which a 50% inhibition of the acetyl chlorinesterase (AChEI) or butyryl cholinesterase (BuCHEI) occurs. Satisfactory inhibiting values—see survey below—are in addition an indication that the compounds of general formula (I), (IA) or (II) are suitable for the production of pharmaceutical agents for the treatment of neurodegenerative diseases, such as Alzheimer's disease.

TABLE 1
Examples of Compounds of General Formulas (I), (IA) and (II) and Results of
Acetyl Cholinesterase and Butyl Cholinesterase Inhibition

| SPH | STRUCTURE | Final AChE | Final BChE | Type |
|---|---|---|---|---|
| SPH-1001 | 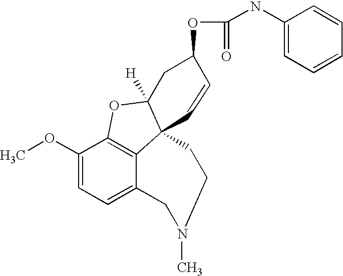 | 200 | 200 | I |
| SPH-1002 | 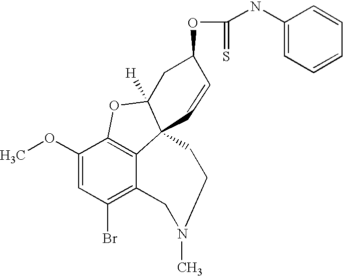 | 45 | 52 | I |
| SPH-1003 | 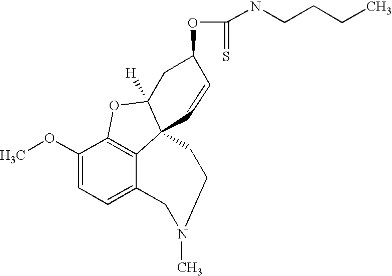 | 200 | 3.8 | I |
| SPH-1005 | 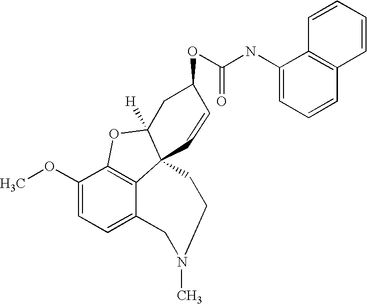 | 200 | 200 | I |
| SPH-1006 | 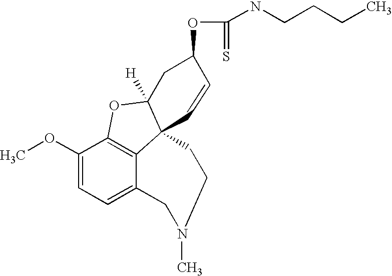 | 200 | 200 | I |
| SPH-1007 | 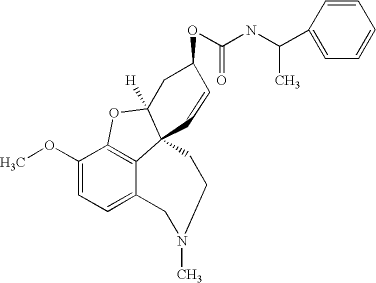 | 200 | 50 | I |
| SPH-1008 | 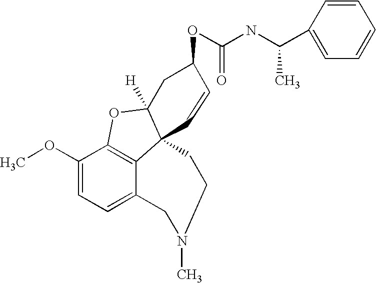 | 94 | 77 | I |
| SPH-1010 | 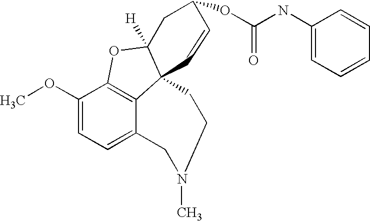 | 90 | 200 | I |
| SPH-1011 | 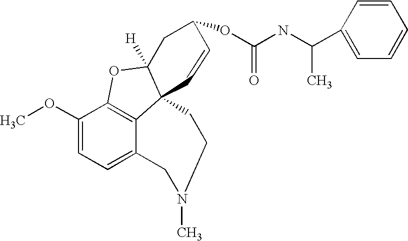 | 75 | 40 | I |
| SPH-1012 | 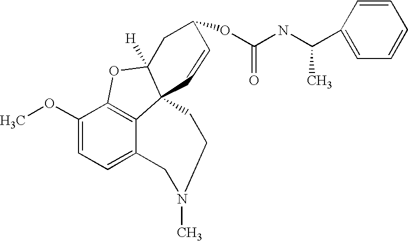 | 70 | 80 | I |
| SPH-1013 | 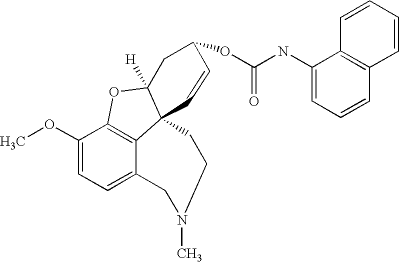 | 200 | 200 | I |
| SPH-1014 | 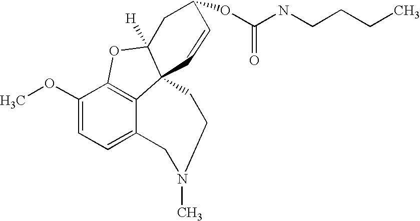 | 200 | 200 | I |
| SPH-1015 | 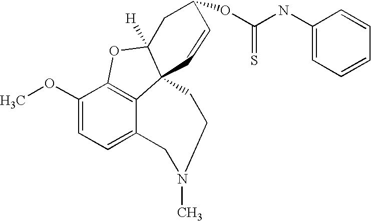 | 30 | 15 | I |
| SPH-1016 | 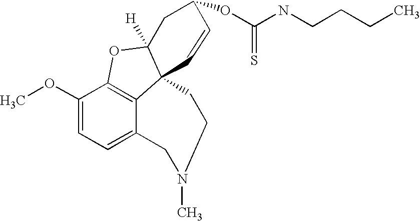 | 40 | 20 | I |
| SPH-1022 | 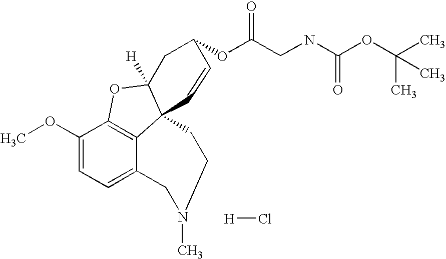 | 200 | 50 | IA |
| SPH-1025 | 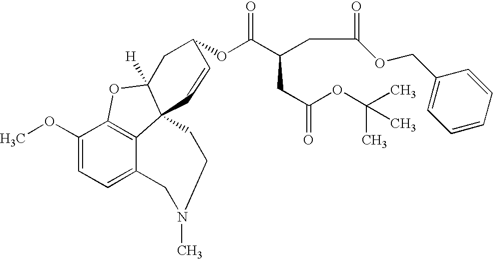 | 18 | 4 | I |
| SPH-1026 | 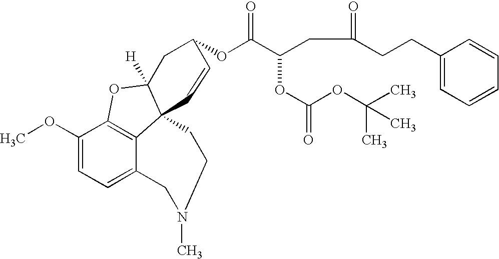 | 11 | 115 | I |
| SPH-1035 | 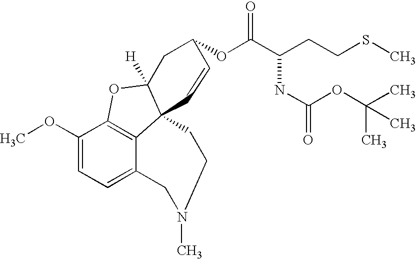 | 4 | 171 | I |
| SPH-1036 | 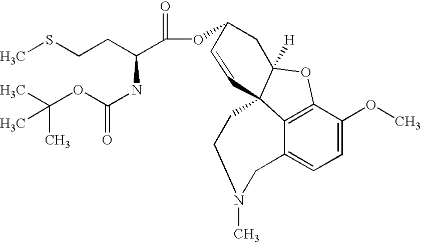 | 16 | 140 | I |
| SPH-1037 | 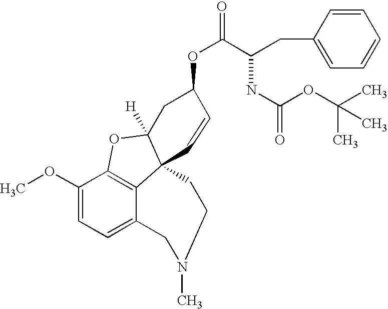 | 19 | 172 | I |
| SPH-1039 | 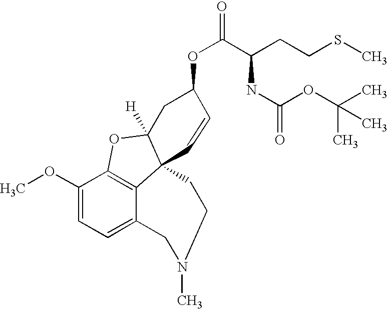 | 15 | 42 | I |
| SPH-1043 | 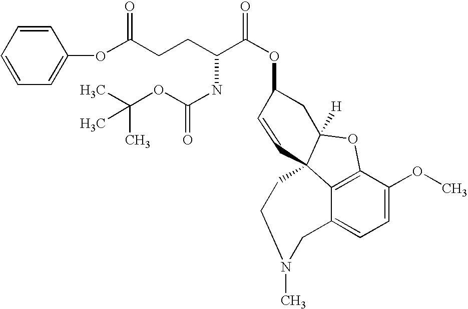 | 19 | 6 | I |
| SPH-1137 | 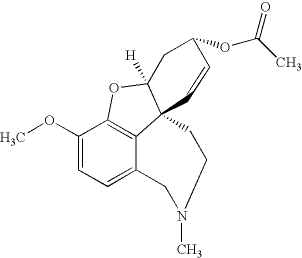 | 200 | 200 | I |
| SPH-1297 | 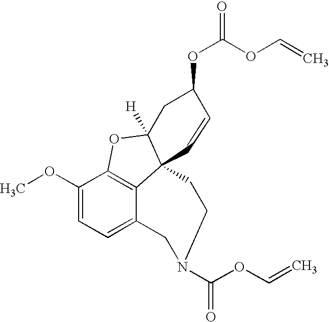 | 200 | 200 | II |
| SPH-1313 | 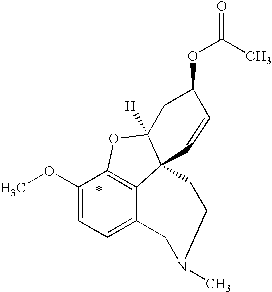 | 31 | 200 | I |
| SPH-1351 | 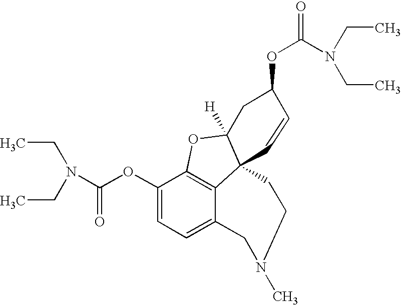 | 11.1 | 16.7 | I |
| SPH-1370 | 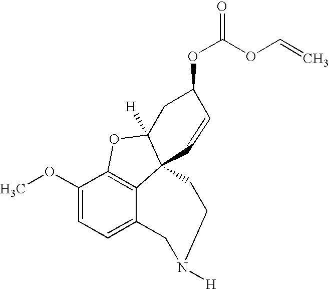 | 17 | 21 | II |
| SPH-1391 | 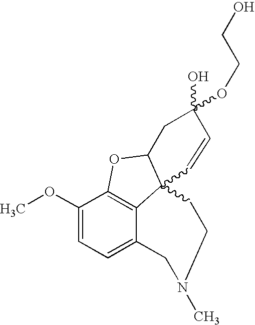 | 18 | 195 | I |
| SPH-1396 | 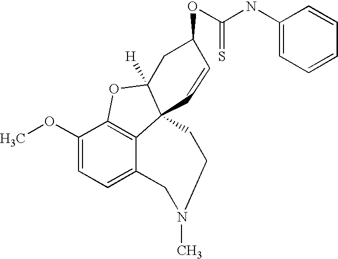 | 51 | 30 | I |
| SPH-1397 | 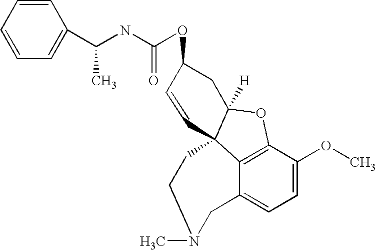 | 10 | 53 | I |
| SPH-1398 | 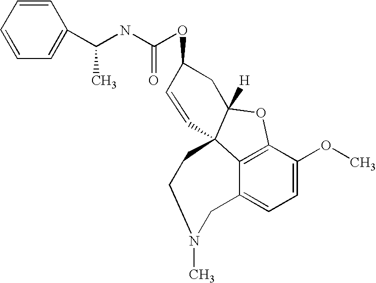 | 16 | 154 | I |
| SPH-1399 | 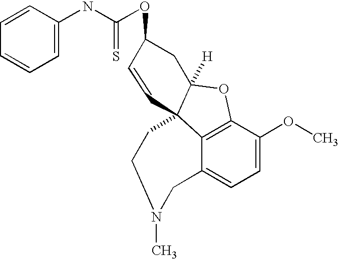 | 19 | 32 | I |
| SPH-1400 | 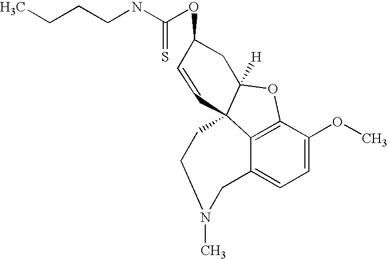 | 19 | 93 | I |
| SPH-1401 | 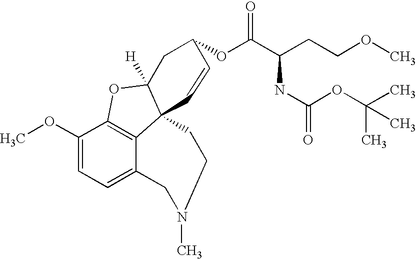 | 10 | 130 | I |
| SPH-1402 | 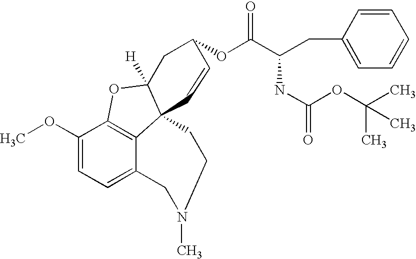 | 16 | 51 | I |
| SPH-1403 | 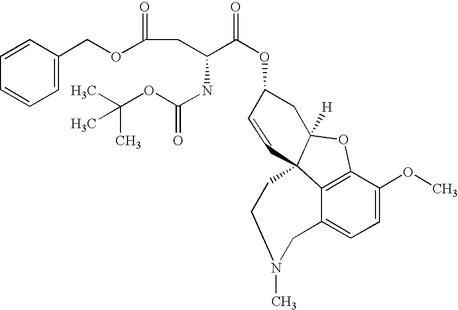 | 6 | 175 | I |
| SPH-1404 | 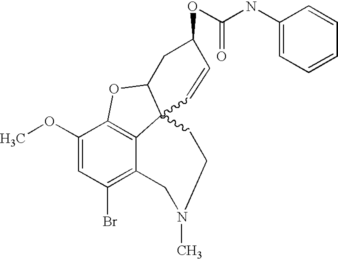 | 7 | 33 | I |
| SPH-1405 | 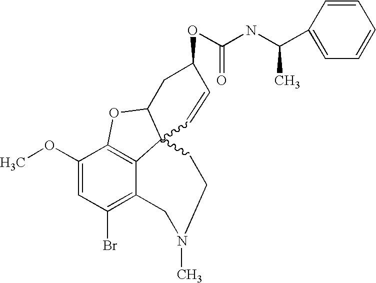 | 5 | 31 | I |
| SPH-1524 | 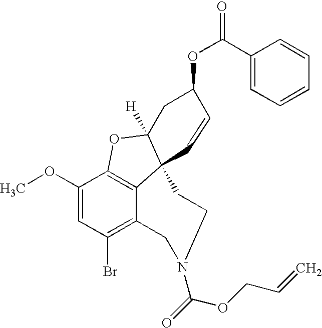 | 1 | 97 | II |
| SPH-1526 | 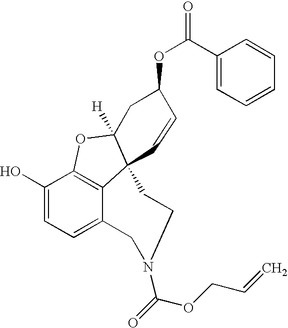 | 11 | 120 | II |
| SPH-1538 | 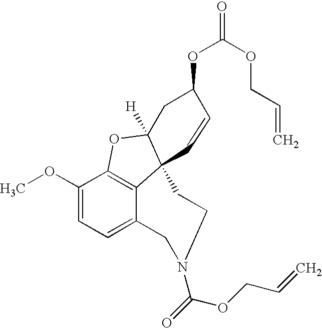 | 1 | 110 | II |
| SPH-1541 | 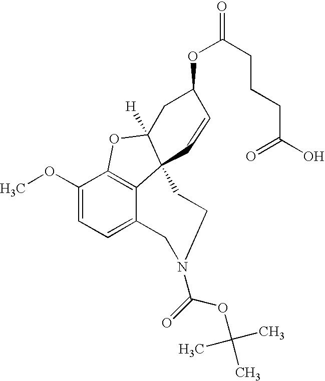 | 0 | 53 | II |
| SPH-1542 | 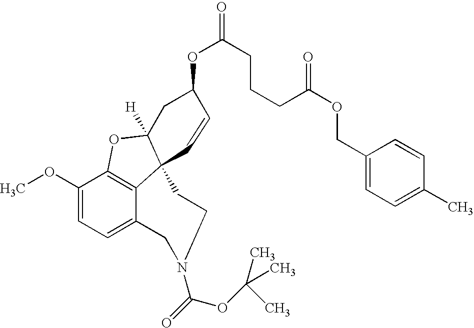 | 8 | 88 | II |
| SPH-3272 | 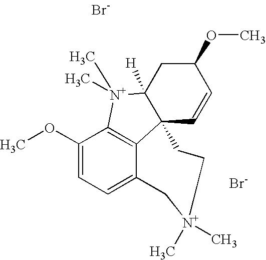 | 18 | 194 | IA |
| SPH-3283 | 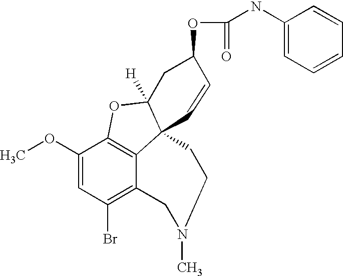 | 18 | 194 | I |
| SPH-3284 | 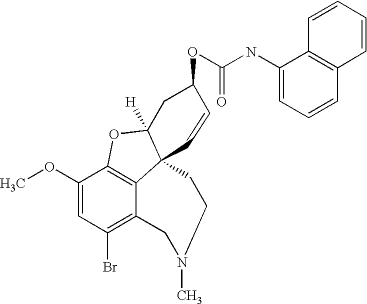 | 4 | 90 | I |
| SPH-3285 | 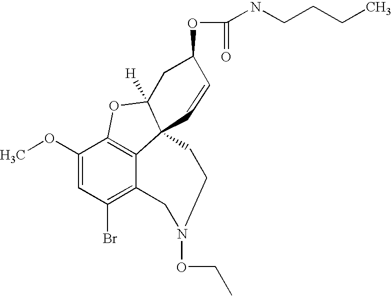 | 2 | 39 | I |
| SPH-3298 | 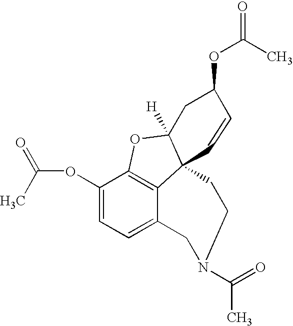 | 12 | 151 | II |
| SPH-3364 | 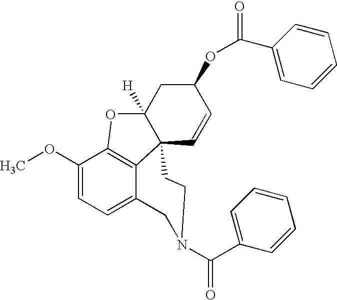 | 16 | 158 | II |
| SPH-3366 | 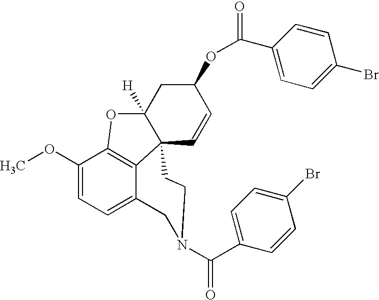 | 19 | 69 | II |
| SPH-3417 | 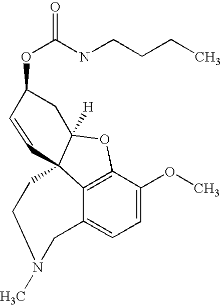 | 200 | 200 | I |

Apart from the above-mentioned, preferred meanings, the substituent R₂ in the general formulas (I), (IA) and (II) can also mean:
i) Hydrogen, a low (C₁-C₁₀, optionally branched or substituted) alkyl group, or cycloalkyl group, a C₃-C₁₀-substituted silyl group (for example triethylsilyl, trimethylsilyl, t-butyldimethylsilyl or dimethylphenylsilyl), a C₂-C₁₀-alpha-alkoxyalkyl group, for example tetrahydropyranyl, tetrahydrofuranyl, methoxymethyl, ethoxymethyl, 2-methoxypropyl, ethoxyethyl, phenoxymethyl or 1-phenoxyethyl;
ii) O—CS—NHR₆ (thiourethanes), in which R₆ has the meanings that are indicated under i);
iii) O—CO—NHR₇ with the following meaning:

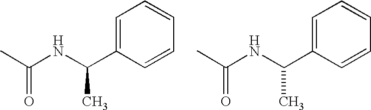

iv) O—CO—HR₆, in which R₆ has the meanings that are mentioned under i), in particular esters with the substitution patterns of amino acids (both enantiomers), such as

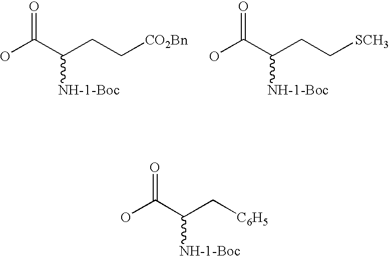

In summary, it can be stated that by the working-up of a debrominated narwedine that is obtained by palladium catalysis according to the invention, namely by contact with oxygen or peroxides, the palladium catalyst that is used can be converted into an insoluble oxide form and separated in a simple way. By this working-up of the reaction mixture, which was completely in line with the safety regulations, it was possible, surprisingly enough, to reduce the palladium radicals to below 5 ppm, so that extremely pure galanthamine or extremely pure galanthamine derivatives could be obtained, which could (can) be used directly in the production of pharmaceutical agents, such as, for example, those for the treatment of Alzheimer's disease.

The compounds, which can be obtained according to the invention, as well as pharmaceutically acceptable acid addition salts thereof, can use active ingredients of pharmaceutical agents for the treatment of neurodegenerative processes, whereby the primary aim is not to bring about an improvement of the acute symptoms and signs, but rather a slowing and modification of the associated processes.

Within the framework of Diabetes mellitus Type II, there is increasing evidence of a role of amyloid fragments in the cell degeneration of the insulin-producing Langerhans islet cells. The cell degeneration can be intensified by a non-controlled calcium ion stream.

The compounds that can be obtained according to the invention as well as pharmaceutically acceptable acid addition salts thereof can be used as active ingredients in pharmaceutical agents, for example for the treatment of degenerative diseases of the islet cells (such as, e.g., Diabetes mellitus Type II).

The compounds that can be obtained according to the invention can be used as active ingredients in pharmaceutical agents, which can be used as follows:
a) For the treatment of Alzheimer's disease,
b) For the treatment of Parkinson's disease,
c) For the treatment of Huntington's disease (chorea),
d) For the treatment of multiple sclerosis,
e) For the treatment of amyotrophic lateral sclerosis,
f) For the treatment of epilepsy,
g) For the treatment of the effects of stroke,
h) For the treatment of the effects of craniocerebral injury,
i) For the treatment and prophylaxis of the effects of diffuse oxygen and nutrient deficiency in the brain, as they are observed after hypoxia, anoxia, asphyxia, cardiac arrest, poisoning, as well as in complications in difficult births in the infant or in anesthesia,
j) Also in particular for prophylactic treatment of apoptotic degeneration in neurons that were or are damaged by local radiotherapy or chemotherapy of brain tumors, and
k) For the treatment of bacterial meningitis, and
l) For the treatment of diseases with apoptotic components, especially in the wake of amyloid-associated cell degeneration,
m) For the treatment of Diabetes mellitus, in particular if it accompanies amyloid degeneration of islet cells,
n) For increasing the muscular strength and the endurance of Alzheimer's patients.

The compounds that can be obtained according to the invention or their pharmaceutically acceptable acid addition salts, e.g., hydrobromide, hydrochloride, methyl sulfate, methiodide, tartrate, fumarate, oxalate, etc. (see table below), can be administered to patients orally, rectally or by subcutaneous, intramuscular, intravenous or intrathecal injection or infusion, or intracerebroventricularly, e.g., by means of an implanted container.

Examples of considered salts of compounds that can be obtained according to the invention are cited in the table below:

| English | Acid | Salt |
|---|---|---|
| Sulfamic | Sulfamic Acid | — |
| | Amidosulfonic Acid | Amidosulfonate |
| 1,2-Ethanedisulfonic | 1,2-Ethanedisulfonic | 1,2-Ethanedisulfonate |
| | Acid | |
| 2-Ethylsuccinic | 2-Ethylsuccinic Acid | 2-Ethylsuccinate |
| 2-Hydroxy- | 2-Hydroxy- | 2-Hydroxy- |
| ethanesulfonic-isethionic | ethanesulfonic Acid | ethanesulfonate |
| 3-Hydroxynaphthoic | 3-Hydroxynaphthoic | 3-Hydroxynaphthoate |
| | Acid | |
| Acetic | Acetic Acid | Acetate |
| Benzoic | Benzoic Acid | Benzoate |
| Benzenesulfonic | Benzenesulfonic Acid | Benzene Sulfonate |
| Calcium | Calcium Dihydrogen | Calcium Ethylene |
| Dihydrogenedetic | Ethylene Diamine | Diamine Tetraacetate |
| | Tetraacetic Acid | |
| Camphorsulfonic | Camphorsulfonic Acid | Camphor Sulfonate |
| Carbonic | Carbonic Acid | Carbonate |
| Citric | Citric Acid | Citrate |
| Dodecylsulfonic | Dodecylsulfonic Acid | Dodecylsulfonate |
| Ethanesulfonic | Ethanesulfonic Acid | Ethanesulfonate |
| Edetic | Ethylenediamine | Ethylenediamine |
| | Tetraacetic Acid | Tetraacetate |
| Fumaric | Fumaric Acid | Fumarate |
| Glubionic | Glubionic Acid | Glubionate |
| Glucoheptonic | Glucoheptonic Acid | Glucoheptonate |
| Gluconic | Gluconic Acid | Gluconate |
| Glutamic | Glutamic Acid | Glutamate |
| Hexylresorcinic | Hexylresorcylic Acid | Hexylresorcylate |
| HBr | Hydrobromic Acid | Hydrobromide |
| HCl | Hydrochloric Acid | Hydrochloride |
| Bicarbonic | Carbonic Acid | Bicarbonate |
| Bitartaric | Tartaric Acid | Bitartrate |
| Hydriodic | Hydriodic Acid | Hydroiodide |
| Lactic | Lactic Acid | Lactate |
| Lactobionic | Lactobionic Acid | Lactobionate |
| Levulinic | Levulinic Acid | Levulinate |
| Estolic (Laurylsulfuric) | Laurylsulfuric Acid | Lauryl Sulfate |
| LIPOIC-(ALPHA) ACID | Lipoic Acid | Liponate |
| Malic | Malic Acid | Malate |
| Maleic | Maleic Acid | Maleinate |
| Malonic | Malonic Acid | Malonate |
| Methanesulfonic | Methanesulfonic Acid | Methanesulfonate |
| Naphthalenesulfonic | Naphthalenesulfonic | Naphthalene Sulfonate |
| | Acid | |
| Nitric | Nitric Acid | Nitrate |
| Pantothenic | Pantothenic Acid | Pantothenate |
| Phosphoric | Phosphoric Acid | Phosphate |
| Polygalacturonic | Polygalacturonic Acid | Polygalacturonate |
| | Pectic Acid | |
| Propionic | Propionic Acid | Propionate |
| Salicylic | Salicylic Acid | Salicylate |
| Succinic | Succinic Acid | Succinate |
| Sulfuric | Sulfuric Acid | Sulfate |
| Tartaric | Tartaric Acid | Tartrate |

Typical dosage rates in the administration of compounds that are obtained according to the invention as active ingredients depend on the nature of the compound that is used and, in the case of intravenous administration, are in the range of 0.01 to 2.0 mg per day and kilogram of body weight based on the physical condition and other medications of the patient.

The following specific formulations can be applied:

Tablets and capsules that contain 0.5 to 50 mg

Solution for parenteral administration that contains 0.1 to 30 mg of active ingredient/ml

Liquid formulations for oral administration in a concentration of 0.1 to 15 mg/ml

Liquid formulations for intracerebroventricular administration in a concentration of 1 or 5 mg of active ingredient/ml.

The compounds can also be a transdermal system in which 0.1 to 10 mg/day is released.

A transdermal metering system consists of a storage layer that contains 0.1 to 30 mg of the active substance as a free base or salt in any case together with a penetration accelerator, e.g., dimethyl sulfoxide or a carboxylic acid, e.g., octanoic acid, and a realistic-looking polyacrylate, e.g., hexyl acrylate/vinyl acetate/acrylic acid copolymer including softeners, e.g., isopropyl myristate. As a cover, an active ingredient-impermeable outer layer, e.g., a metal-coated, siliconized polyethylene patch with a thickness of, for example, 0.35 mm, is used. To produce an adhesive layer, e.g., a dimethylamino methacrylate/methacrylate copolymer in an organic solvent is used.

In particular, the compounds that are obtained according to the invention, which in many cases show a cholinesterase-inhibiting action, are suitable as therapeutic and/or prophylactic active ingredients for senile dementia, Alzheimer's disease, etc. The compounds that can be obtained according to the invention are new, extremely pure forms of tetracyclic, condensed, heterocyclic compounds.

In summary, an embodiment of the invention can be represented as follows:

The invention relates to a process for the production of extremely pure galanthamine or extremely pure galanthamine derivatives, whereby a start is made from racemic bromine narwedine, which is debrominated under palladium catalysis. In this case, the working-up of the reaction mixture, which is carried out in the presence of oxygen or peroxides so that the palladium catalyst in an insoluble form is converted into an easily separable form, is essential to the invention. The further reaction is carried out by reduction of enantiomer-pure narwedine to form enantiomer-pure galanthamine, whereby it is then alkylated or dealkylated so that a corresponding substitution on the ring-nitrogen atom is achieved. By further purification, such as recrystallization, residual portions of palladium of below 5 ppm are achieved, so that direct use as a pharmaceutical raw material is made possible.

1. Process for the production of extremely pure 4a,5,9,10,11,12,-hexahydro-6H-benzofuro[3a,3,2-ef][2]benzazepine derivatives of general formulas I, IA and II: 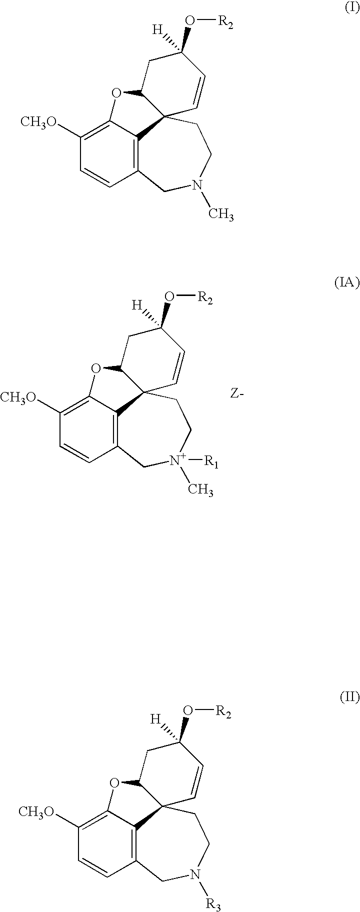
in which R1 is selected from the group that consists of hydrogen, hydroxy, alkoxy, low alkyl(C2-C10), which optionally is substituted by at least one halogen, low alkenyl(C2-C10), aryl, aralkyl, aryloxyalkyl; R2 is selected from the group that consists of low alkyl(C2-C10), which optionally is substituted by at least one halogen, low alkenyl(C2-C10), low alkinyl(C2-C10), aryl, aralkyl, aryloxyalkyl, alkylcarbonyl, arylcarbonyl, aralkylcarbonyl, alkyloxycarbonyl, aryloxycarbonyl, aralkyloxycarbonyl, alkylthionyl, arylthionyl, aralkylthionyl, alkyloxythionyl, aryloxythionyl, aralkyloxythionyl, alkylsulfonyl, aralkylsulfonyl, arylsulfonyl, carboxamide, thiocarboxamide; R3 is selected from the group that consists of hydrogen, hydroxy, alkoxy, low alkyl(C2-C10), which optionally is substituted by at least one halogen, low alkenyl(C2-C10), low alkinyl(C2-C10), aryl, aralkyl, aryloxyalkyl, formyl, alkylcarbonyl, arylcarbonyl, aralkylcarbonyl, alkyloxycarbonyl, aryloxycarbonyl, aralkyloxycarbonyl, alkylsulfonyl, aralkylsulfonyl, arylsulfonyl, and whereby Z is an anion of a pharmaceutically acceptable organic acid or an inorganic anion, characterized in that in a reaction step 1, racemic bromine narwedine (II) is debrominated with palladium (II) acetate and triphenylphosphine; in a reaction step 2, the reaction mixture that contains racemic narwedine (IV) is worked up under oxygen contact and is converted to enantiomer-pure narwedine (V); and whereby in a reaction step 3, enantiomer-pure galanthamine of formula (VI) is obtained by reduction, and in a reaction step 4, compounds of general formula (I) are obtained by O-alkylation or in a reaction step 4′, compounds of general formula (IA) are obtained by O-alkylation as well as subsequent salt formation, or in a reaction step 4″, compounds of general formula (II) are obtained by O-alkylation, N-demethylation and N-alkylation.

2. Process according to claim 1, wherein the oxygen contact in reaction step 2 is carried out with an air-nitrogen mixture.

3. Process according to claim 2, wherein the air-nitrogen mixture contains 0.2 to 20% by volume of oxygen.

4. Process according to claim 1, wherein the oxygen contact is carried out in the presence of activated carbon.

5. Process according to claim 1, wherein the reaction step 3 and/or the reaction step 4 is (are) downstream to one or more purification step(s), preferably recrystallization.

6. Process for the production of extremely pure 4a,5,9,10,11,12,-hexahydro-6H-benzofuro[3a,3,2-ef][2]benzazepine derivatives with general formulas I, IA and II 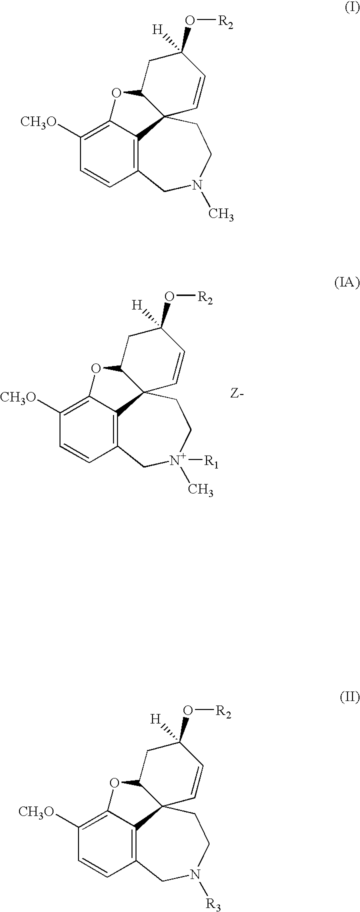
in which R1 is selected from the group that consists of hydrogen, hydroxy, alkoxy, low alkyl(C2-C10), which optionally is substituted by at least one halogen, low alkenyl(C2-C10), aryl, aralkyl, aryloxyalkyl; R2 is selected from the group that consists of low alkyl(C2-C10), which optionally is substituted by at least one halogen, low alkenyl(C2-C10), low alkinyl(C2-C10), aryl, aralkyl, aryloxyalkyl, alkylcarbonyl, arylcarbonyl, aralkylcarbonyl, alkyloxycarbonyl, aryloxycarbonyl, aralkyloxycarbonyl, alkylthionyl, arylthionyl, aralkylthionyl, alkyloxythionyl, aryloxythionyl, aralkyloxythionyl, alkylsulfonyl, aralkylsulfonyl, arylsulfonyl, carboxamide, thiocarboxamide; R3 is selected from the group that consists of hydrogen, hydroxy, alkoxy, low alkyl(C2-C10), which optionally is substituted by at least one halogen, low alkenyl(C2-C10), low alkinyl(C2-C10), aryl, aralkyl, aryloxyalkyl, formyl, alkylcarbonyl, arylcarbonyl, aralkylcarbonyl, alkyloxycarbonyl, aryloxycarbonyl, aralkyloxycarbonyl, alkylsulfonyl, aralkylsulfonyl, arylsulfonyl, and whereby Z is an anion of a pharmaceutically acceptable organic acid or an inorganic anion, characterized in that in a reaction step 1, racemic bromine narwedine (II) is debrominated with palladium (II) acetate and triphenylphosphine; in a reaction step 2, the reaction mixture, containing racemic narwedine (IV) is worked up with use of peroxides and is converted to enantiomer-pure narwedine (V), and whereby in a reaction step 3, enantiomer-pure galanthamine of general formula (VI) is obtained by reduction, and in a reaction step 4, compounds of general formula (I) are obtained by O-alkylation or in a reaction step 4′, compounds of general formula (IA) are obtained by O-alkylation as well as subsequent salt formation, or in a reaction step 4″, compounds of general formula (II) are obtained by O-alkylation, N-demethylation and N-alkylation.

7. Process according to claim 6, wherein in reaction step 2, inorganic peroxides, preferably hydrogen peroxide, are used as peroxides.

8. Process according to claim 6, wherein in reaction step 2, organic peroxides, preferably metachloroperbenzoic acid, are used as peroxides.

9. Process according to claim 6, wherein activated carbon in addition to the peroxides is also present in reaction step 2.

10. Process according to claim 6, wherein one or more purification step(s), preferably recrystallization, is (are) downstream to reaction step 3 and/or reaction step 4.

11. Process according to claim 2, wherein the oxygen contact is carried out in the presence of activated carbon.

12. Process according to claim 3, wherein the oxygen contact is carried out in the presence of activated carbon.

13. Process according to claim 2, wherein the reaction step 3 and/or the reaction step 4 is (are) downstream to one or more purification step(s), preferably recrystallization.

14. Process according to claim 3, wherein the reaction step 3 and/or the reaction step 4 is (are) downstream to one or more purification step(s), preferably recrystallization.

15. Process according to claim 4, wherein the reaction step 3 and/or the reaction step 4 is (are) downstream to one or more purification step(s), preferably recrystallization.

16. Process according to claim 7, wherein activated carbon in addition to the peroxides is also present in reaction step 2.

17. Process according to claim 8, wherein activated carbon in addition to the peroxides is also present in reaction step 2.

18. Process according to claim 7, wherein one or more purification step(s), preferably recrystallization, is (are) downstream to reaction step 3 and/or reaction step 4.

19. Process according to claim 8, wherein one or more purification step(s), preferably recrystallization, is (are) downstream to reaction step 3 and/or reaction step 4.

20. Process according to claim 9, wherein one or more purification step(s), preferably recrystallization, is (are) downstream to reaction step 3 and/or reaction step 4.
